(19) United States
(12) Patent Application Publication
(10) Pub. No.: US 2003/0161975 A1
(43) Pub. Date: Aug. 28, 2003

(54) Polyisoprene condom

(76) Inventors: David M. Lucas, Uniontown, OH (US); Jorge V. Virreira, Canton, OH (US); Paul Cacioli, Canton, OH (US)

Correspondence Address: PATENT DOCKET DEPARTMENT, GARDNER CARTON & DOUGLAS LLC, 191 N. WACKER DRIVE, SUITE 3700, CHICAGO, IL, 60606, US

(21) Appl. No.: 10/085,890

(22) Filed: Feb. 27, 2002

(51) Int. Cl. B32B 1/08; B65D 1/00; B29D 22/00; B29D 23/00; F16L 1/00; A61F 5/44

(52) U.S. Cl. 428/35.5; 428/36.9; 428/36.8; 604/349

(57) ABSTRACT

An article, particularly a glove or condom, of polyisoprene that is cross-linked with a curing package that comprises diisopropyl xanthogen or xanthogen disulfide. The glove or condom surface is smooth, continuous and defect-free as viewed with a scanning electron microscope. The articles demonstrate improved latex stability over polyisoprene articles currently in use.

### FIELD OF THE INVENTION

The invention relates to a condom of polyisoprene polymer and curing compounds useful in curing polyisoprene formulations.

### BACKGROUND OF THE INVENTION

Condoms are typically made from vulcanized natural rubber. Natural rubber is produced in latex form by the Hevea Brasiliensis tree and has unique characteristics. These characteristics make natural rubber particularly useful for the preparation of barrier protection products. Among the unique characteristics is its high level of stereo regularity, meaning that the polymer chain consists almost exclusively of cis-1,4 isoprene units. Natural rubber latex is also a highly branched polymer with a high molecular weight and a wide molecular weight distribution. These characteristics of the base latex result in vulcanized rubber film products having a unique combination of strength and elasticity. However, natural polyisoprene also contains proteins that have been shown to produce dermal allergic reaction in some susceptible individuals.

Synthetic polyisoprene has been developed to provide a material with the benefits of natural rubber and eliminate the potential for protein allergy. However, development of a true replacement for natural rubber has proved difficult, with synthetic variants typically having a lower level of stereo regularity and different molecular weight characteristics. This development, in turn, has resulted in synthetic polyisoprene films that have an inferior balance of properties than those of a vulcanized natural rubber film.

In dip molding processes, the majority of work with synthetic polyisoprene has focussed on the development of polyisoprene gloves, using a coagulation dip process. In this type of process, a glove-shaped mold is first dipped into a solution that is known to destabilize the latex formulation. This coagulant layer is then dried, before the mold is dipped into a bath of the compounded latex formation. The coagulated wet gel would typically be leached in water to remove residual surfactant before being dried at a relatively high temperature to complete the crosslinking of the rubber film.

One prior art patent includes the preparation of polyisoprene gloves and condoms using standard compounding admixtures such as sulfur, zinc oxide, organic accelerators, stabilizers, waxes, anti-aging substances, viscosity regulators, fillers, and pigments. However, the process disclosed in the patent is primarily focused at creating a coating on the elastomeric article, which has surface deviations such that the deviations impart slip between the user and the elastomeric article.

Another prior art patent describes the preparation of hypoallergenic rubber products which shrink from a second shape and size to their original shape and size on application of heat. The examples include a polyisoprene condom which will shrink to fit the individual user during use. The curing package used to make this condom was known in the art and consisted of agents such as peroxides and/or sulfur.

Another prior art polyisoprene article used a combination of sulfur, zinc oxide and dithiocarbamate as a curing package. However, the latex showed poor shelf-stability, typically coagulating within a few days of compounding.

There is a need, therefore, for a polyisoprene condom that exhibits a continuous, defect-free film, which does not allow penetration of micro-organisms or sperm. There is further a need for a polyisoprene latex condom that shows no deterioration in physical properties of the dipped film upon maturation of the compounded latex formulation, and a need for a condom that will maintain its physical integrity on aging. There is further a need for a polyisoprene condom that is produced without a coagulant step and, therefore, allows for a thinner film product.

### SUMMARY OF THE INVENTION

Accordingly, the present invention provides a synthetic polyisoprene condom that is dipped from a formulated latex and show good stability, exhibits no deterioration in physical properties upon maturation of the formulated latex, has a continuous defect-free surface and show retention of physical properties on aging. The condom is formed in a straight dip process, meaning that no coagulation step has taken place prior to drying of the latex film.

The polyisoprene articles of the present invention are produced with curing compounds that are combinations of sulfur, xanthates and thiurams.

### BRIEF DESCRIPTION OF THE DRAWINGS

FIG. 1 is a scanning electron micrograph image of the surface of a polyisoprene condom prepared according to the present invention and including accelerator AS100 with tetraethyl thiuram disulfide curing package.

FIG. 2 is a scanning electron micrograph image of the surface of a polyisoprene condom prepared with a curing package including zinc diethyl dithiocarbamate (“ZDEC”) and zinc dibutyl dithiocarbamate (“ZBDC”).

### DETAILED DESCRIPTION OF THE INVENTION

Embodiments of the present invention provide a synthetic polyisoprene latex condom. The latex is preferably cured with a combination of sulfur, diisopropyl xanthogen polysulfide (available as AS100 from Akron Dispersions, Akron, Ohio) and tetrabenzyl thiuram disulfide (“TBzTD,” available from Akrochem, Akron, Ohio). Other xanthate and thiuram combinations may also be used including, but not limited to, dibutyl xanthogen disulphide (available from Uniroyal Chemical as “CPB”), tetraethyl thiuram disulfide (“TETD,” available from Akron Dispersions) and dipentamethylene thiuram hexasulfide. Other conventional natural rubber accelerator systems can be used and these include zinc dithiocarbamates such as zinc dibenzyl dithiocarbamate (“ZBEC”). However, the acceptable dipping life of the compounded latex is significantly shortened with the use of such systems.

Details of three formulations prepared in accordance with the present invention are shown in Table 1.

TABLE 1
Synthetic Polyisoprene Latex Formulations.

| | | Concentration of total formulation, (phr) | | |
|---|---|---|---|---|
| Chemical name | Supplier, location | A | B | C |
| Synthetic cis-1,4-polyisoprene | Kraton Polymers, Houston, TX | 100 | 100 | 100 |
| Sulfur | | 1.5 | 1.5 | 1.5 |
| Diisopropyl xanthogen | Akron Dispersions, Akron, OH | 1.5 | 1.5 | 1.5 |
| polysulphide (“AS100”) | | | | |
| Tetrabenzyl thiuram disulfide | Akrochem Corp, Akron, OH | 0.6 | | |
| Tetraethyl thiuram disulfide | Akrochem Corp | | 0.6 | |
| Zinc dibenzyl dithiocarbamate | Akrochem Corp | | | 0.4 |
| Potassium oleate | Akrochem Corp. | 0.4 | 0.4 | 0.4 |
| Ethoxylated cetyl/stearyl | Cognis Corporation, | 0.15 | 0.15 | 0.15 |
| alcohol | Cincinnati, OH | | | |
| Wingstay L | Akron Dispersions | 0.5 | 0.5 | 0.5 |
| Potassium hydroxide | | pH 10.5-10.8 | pH 10.5-10.8 | PH 10.5-10.8 |
| (pH adjustment) | | | | |

Alternative embodiments of the present invention include formulations with curing compounds that, in addition to sulfur and diisopropyl xanthogen polysulfide, include diisopropyl xanthogen, diisopropyl xanthogen plus tetraethylthiuram disulfide, diisopropyl xanthogen plus zinc dibenzyldithiocarbamate, xanthogen sulfide, xanthogen sulfide plus tetraethylthiuram disulfide, and xanthogen sulfide plus zinc dibenzyldithiocarbamate. Alternative embodiments of the present invention may also include curing compounds that that comprise sulfur, a thiuram compound and diisopropyl xanthogen, diisopropyl xanthogen plus tetraethylthiuram disulfide, diisopropyl xanthogen plus zinc dibenzyldithiocarbamate, xanthogen sulfide, xanthogen sulfide plus tetraethylthiuram disulfide, and xanthogen sulfide plus zinc dibenzyldithiocarbamate.

The alternative formulations that may result in condoms having a continuous defect-free polyisoprene film have a concentration of tetrabenzyl thiuram disulfide, tetraethyl thiuram disulfide, or zinc dibenzyl dithiocarbamate that varies by +/−25%.

The compounded latex used in the dipping process had the characteristics summarized in Table 2.

TABLE 2
Latex Characteristics.

| | |
|---|---|
| Viscosity, centipoise | 90-100 |
| PH | 10.5-10.8 |
| Total Solids content, % by weight | 50-60 |
| Temperature | 77° F. |

The dipping process was performed in the following steps:

1. Smooth glass formers were cleaned and dried.

2. Formers were dipped in the first latex bath.

3. The first latex film was dried in a conventional oven at 140° F. for 3-4 minutes.

4. The film was cooled to room temperature or below.

5. The formers were dipped in a second latex bath.

6. The second film was dried in a conventional oven at 140° F. for 3-4 minutes.

7. A ring was formed on the condom.

8. The condoms were leached with water at 140° F. or higher for 1 minute.

9. The latex was cured at 300° C. for 5 minutes.

10. The condoms were leached with water at 140-150° F. for 1 minute.

11. The condoms were stripped from the former with powder.

Condoms prepared by this process were characterized for 500% modulus, tensile strength, and elongation according to ASTM D412, Test Methods for Vulcanized Rubber. A summary of the physical properties of the condoms characterized by the foregoing methods appears in Table 3.

TABLE 3
Summary of Condom Characteristics as Prepared
Using Formulations A-C.

| | 500% Modulus, | Tensile strength, | Elongation, |
|---|---|---|---|
| Formulation | Mpa | MPa | % |
| A | 2.0 | 27 | >1050 |
| B | 2.0 | 20 | >1050 |
| C | 1.0 | 15 | >1050 |

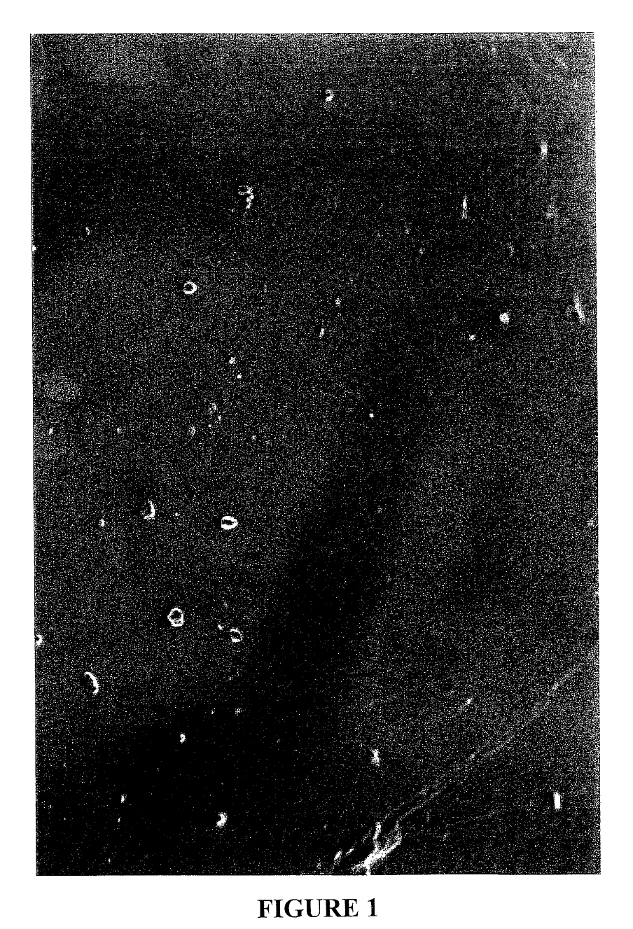

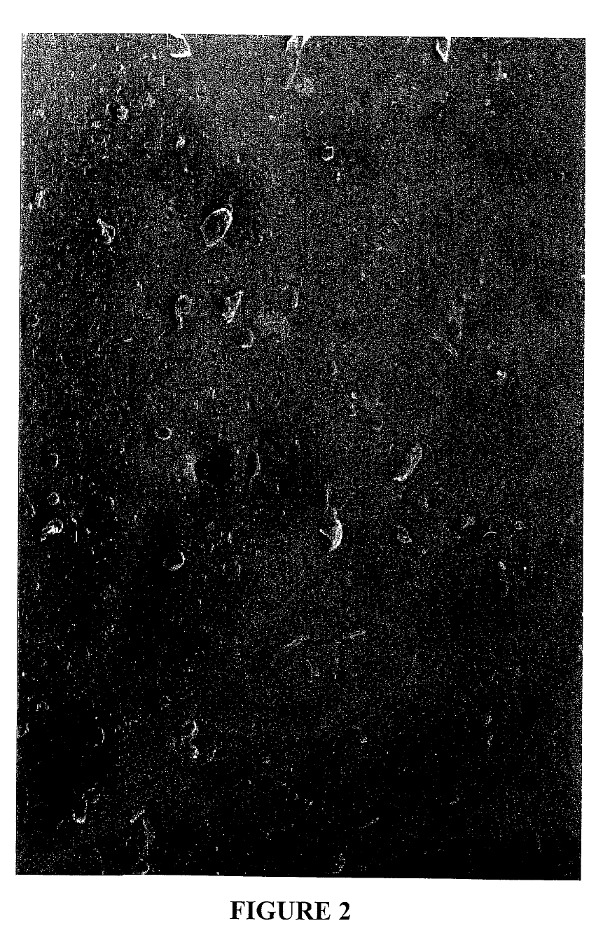

Condoms formed using Formulation A and a formulation including ZDEC and ZBDC were studied using a Hitachi S-3000N scanning electron microscope using secondary electron image resolution. The micrograph images produced from this study revealed a continuous, defect-free film of a condom prepared with Formulation A as shown in FIG. 1. FIG. 2 depicts the surface of a condom prepared from a polyisoprene formulation including ZDEC and ZBDC. Visible in FIG. 2 are divots that are representative of the type of defect commonly seen on the surface thin films prepared with polylisoprene formulations.

The formulations of the present invention may be used to make articles other than condoms where a thin film of this type is desirable.

It should be understood that various changes and modifications to the embodiments described herein will be apparent to those skilled in the art. Such changes and modifications can be made without departing from the spirit and scope of the present invention and without demising the attendant advantages. It is, therefore, intended that such changes and modifications be covered by the appended claims.

What is claimed is:

1. A polyisoprene article comprising,
polyisoprene latex; and
a curing compound comprising sulfur, a thiuram compound and a second compound selected from a group consisting of diisopropyl xanthogen polysulfide, diisopropyl xanthogen, diisopropyl xanthogen plus tetraethylthiuram disulfide, diisopropyl xanthogen plus zinc dibenzyldithiocarbamate, xanthogen sulfide, xanthogen sulfide plus tetraethylthiuram disulfide, and xanthogen sulfide plus zinc dibenzyldithiocarbamate.

2. The article of claim 1 wherein the thiuram compound is tetrabenzyl thiuram disulfide.

3. The article of claim 1 wherein the polyisoprene is cis-1,4-polyisoprene.

4. The article of claim 1 wherein the thiuram compound is present in a concentration range of between about 0.45 parts per 100 parts of polyisoprene and about 0.75 parts per 100 parts of polyisoprene.

5. The article of claim 4 wherein the thiuram compound is present in a concentration of about 0.6 parts per 100 parts of polyisoprene.

6. The article of claim 1 wherein the second compound is zinc dibenzyldithiocarbamate.

7. The article of claim 6 wherein the zinc dibenzyldithiocarbamate is present in a concentration range of between about 0.3 parts per 100 parts of polyisoprene and about 0.5 parts per 100 parts of polyisoprene.

8. The article of claim 7 wherein the zinc dibenzyldithiocarbamate is present in a concentration range of about 0.4 parts per 100 parts of polyisoprene.

9. The article of claim 1 wherein the second compound containing xanthogen is present in the article in a concentration range of between about 1.0 and about 2.0 parts per 100 parts of polyisoprene.

10. The article of claim 9 wherein the second compound containing xanthogen is present in the article at a concentration of about 1.5 parts per 100 parts of polyisoprene.

11. The article of claim 1, wherein said article is a condom.

12. The article of claim 11, wherein said condom is a continuous, defect-free film.
